(19) United States
(12) Patent Application Publication
(10) Pub. No.: US 2006/0052262 A1
(43) Pub. Date: Mar. 9, 2006

(54) Perfluoropolyether compound and lubricant and magnetic disk using same

(75) Inventors: Tamio Akada, Kobe-shi (JP); Nagayoshi Kobayashi, Kobe-shi (JP); Yoshinobu Fujii, Kobe-shi (JP)

Correspondence Address: KUBOVCIK & KUBOVCIK, SUITE 710, 900 17TH STREET NW, WASHINGTON, DC, 20006, US

(73) Assignee: MATSUMURA OIL RESEARCH CORP., Kobi-shi (JP)

(21) Appl. No.: 10/529,849

(22) PCT Filed: Oct. 1, 2003
(86) PCT No.: PCT/JP03/12570

§ 371 Date: Mar. 31, 2005

(30) Foreign Application Priority Data
Oct. 1, 2002 (JP) 2002-324525

(51) Int. Cl. C10M 105/58; C07C 217/28

(52) U.S. Cl. 508/556; 508/562; 508/563; 564/505; 428/800

(57) ABSTRACT

A compound of the formula (I), a lubricant and a magnetic disk using the compound
A—CH₂CF₂O(CF₂CF₂O)m-(CF₂O)n-CF₂CH₂—B (I) wherein A is amino group having hydroxyl group, B is hydroxyl group, or amino group having or not having hydroxyl group, wherein m is a real number of 5 to 36, and n is a real number of from 4 to 30.

### TECHNICAL FIELD

The present invention relates to a fluorine compound containing amino group and hydroxyl group, a lubricant containing the fluorine compound having amino group and hydroxyl group, and a magnetic disk produced using the compound. More specifically, the invention concerns with a compound for a lubricant used in forming a recording medium such as a magnetic disk or a magnetic tape, i.e., a large-volume recording medium, and a lubricant containing the compound.

### BACKGROUND ART

With an increase in recording density of magnetic disks, the distance between a magnetic disk for use as a recording medium and a head for recording/reproduction of information is so narrowed as to nearly come into contact. A lubrication layer is formed on the surface of the magnetic disk to prevent the disk from wearing by contact and/or sliding against the head. The lubrication layer is usually formed by applying a lubricant to the surface of the magnetic disk.

Generally perfluoropolyether is used as such lubricant. Among various lubricants, chiefly used is a Fomblin-based compound (produced by Ausimont S.p.A.) which is low in adhesion to a head and in frictional force. The basic skeleton of Fomblin-based compound is perfluoropolyether wherein the repeated unit is (CF₂CF₂O)m-(CF₂O)n-.

However, Fomblin-based perfluoropolyether is such that Al₂O₃ in the material of the head is reacted as Lewis acid with oxygen atom in perfluoropolyether chain to cleave the chain (e.g. nonpatent literature 1). With the progress of this cleavage, Fomblin-based perfluoropolyether is made into a low molecular weight compound, and is eventually evaporated from the surface of the magnetic disk.

X-1P produced by The Dow Chemical Co. is known as an additive for inhibiting the decomposition of Fomblin-based perfluoropolyether. X-1P has a cyclophosphazene ring in a molecule. Fluorophenoxy group and trifluoromethylphenoxy group (numbering 6 in total) are bonded to the ring.

The effect of X-1P in preventing decomposition of a lubricant is achieved as follows. The cyclophosphazene ring interacts with Al₂O₃ prior to perfluoropolyether to thereby inhibit decomposition of perfluoropolyether(e.g. nonpatent literature 2). However, X-1P is dissolved in a certain degree in a solvent wherein the basic structure is perfluorobutane with methoxy group or ethoxy group at an end (e.g. HFE-7100 or HFE-7200 produced by 3M). But X-1P is not dissolved in perfluorocarbon-based solvent conventionally used in applying a lubricant. Further, X-1P is low in compatibility with perfluoropolyether. Because of such properties, a phase separation occurs in the lubrication layer on the surface of magnetic disks, resulting in failure to form a uniform lubrication layer.

In using an additive such as X-1P, various problems arise in producing disks coated with a lubricant. E.g., usable solvents may be limited as described above. When a lubricant is applied to the disk by a dip coat method, adsorbability to the disk may be different depending on the main lubricant and the additive, and the concentrations of these compounds in the solvents in the bath are changed with time. If the components in the bath show different concentrations, the lubrication layer on the disk indicates different component ratio, whereby problems are presented in that quality control of the disk becomes difficult and complicated.

On the other hand, a magnetic disk apparatus mainly operates on a contact/start/stop system (CSS system) for starting and stopping a drive device. In the CSS system, a magnetic head is brought into contact with a disk in starting and stopping the drive device. However, in such magnetic disk apparatus, a high adhesive strength is imposed between the disk and the head in starting the disk so that the disk may be likely to fail to restart the disk due to a high adsorbability when restarting the disk after a long-time contact of the head with the disk. To avoid this problem, a lamp/load/unload (L/UL) system is adopted in which the head is retracted from the surface of the disk in stopping the disk.

In recent years, disk apparatuses have been increasingly improved in recording density and in a high-speed processing. To cope with the improvements, it has become necessary to shorten (lower) the distance (floating quantity) between the head and the disk or to increase the rotational speed of the disk. Even in the L/UL system wherein the head is not contacted with the disk, the frequency of contact of the head with the disk is increased in view of lower floating quantity or high-speed processing and the lubricant may be transferred from the surface of the disk to the head and may be scattered outside the surface of the disk, whereby the lubrication layer may be eliminated, resulting in tendency to damage the disk. Therefore, it is desired to use a lubricant having high adhesion to the surface of the disk in the L/UL system.

One of such lubricants is Fomblin-based perfluoropolyether having a plurality of polar groups in a molecule (Fomblin Zdol, Fomblin Z-Tetraol, etc.). However, it is impossible to avoid the following phenomenon. As the result of reaction with Al₂O₃, there occur cleavage of perfluoropolyether chain and decomposition, so that perfluoropolyether is eventually evaporated from the surface of the disk.

A compound wherein an end of perfluoropolyether is alkylamine is disclosed as the compound which is inhibited from decomposition with Al₂O₃, despite the presence of perfluoropolyether chain in a molecule (e.g. patent literature 1). Also disclosed is perfluoropolyether-based lubricant which scarcely moves toward the head and which has amino group at an end (e.g. patent literature 2). The amino groups exemplified in the foregoing patent literature are those derived from an aliphatic, alicyclic or aromatic amine compound. However, the compound contains little or no polar group component which contributes to adhesion to the disk surface. This defect is a problem to be overcome in a disk apparatus in which the disk is rotated at a high speed.

As set forth hereinbefore, the improvements in the performance of lubricants and magnetic disks have increased the importance more than ever for the purpose of improving the performance of magnetic disk apparatus.

[Nonpatent Literature 1]

Macromolecules, 1992, vol.25, pp. 6791-6799

[Nonpatent literature 2]

Proceedings of The Micromechanics for Information and Precision Equipment Conference, Tokyo, Japan, 1997, pp.367-370 [Patent literature 1] Specification of US Pat. No.6,083,600 [Patent literature 2] JP 172268/1999 A

Currently chiefly used as the lubricant for magnetic disks are derivatives wherein the basic skeleton is perfluoropolyether having a low scattering degree and a good sliding property. The rotational frequency of the disk for high-speed recording and reproduction of information will be very likely to reach 15000 rpm or more in near future. As the rotational frequency of the disk increases, the lubricant tends to be scattered in a higher degree. This is caused not only by the increase in rotational speed of disks with an increase in rotational frequency thereof but also by an elevation of temperature of the apparatus in its entirety.

When sliding is continued for a long time at a high rotational frequency, the lubricant may become decomposed. In this case, the lubrication layer may become thinner, so that the apparatus is unlikely to exhibit a sufficient sliding property, leading to damage of the recording layer. Further, if the surface of the disk adheres to the head in a high degree, too heavy a load may be carried on a spindle motor for rotating the disk, whereby finally the rotation of disks will come to a halt. If the disk is forcedly rotated by a spindle motor with a large torque, the head may be broken and the disk may be damaged. The damage of disks may result in breakage of the recording layer so that a lubricant should have high adsorbability to the disk and would be unlikely to be decomposed to assure the reliability of the apparatus.

It is important to develop a lubricant which can be used singly to avert cumbersome procedure involved in producing a disk using an additive in combination.

To overcome the foregoing problems, a variety of compounds were prepared and their properties were investigated.

As a result, it was found that a compound wherein the end of perfluoropolyether chain is amino group having hydroxyl group can overcome the above-mentioned problems.

It was also found that magnetic disks using the compound for the lubrication layer are suitable for a disk apparatus in which magnetic disks are rotated at a high rotational frequency.

### DISCLOSURE OF THE INVENTION

The present invention relates to the following.

(1) A compound of the formula (I)
A—CH₂CF₂O(CF₂CF₂O)m-(CF₂O)n-CF₂CH₂—B (I)
wherein A is amino group having hydroxyl group, B is hydroxyl group, or amino group having or not having hydroxyl group, wherein m is a real number of 5 to 36, and n is a real number of from 4 to 30.
(2) A lubricant containing the compound of the formula (I)
A—CH₂CF₂O(CF₂CF₂O)m-(CF₂O)n-CF₂CH₂—B (I)
wherein A is amino group having hydroxyl group, B is hydroxyl group, or amino group having or not having hydroxyl group, wherein m is a real number of 5 to 36, and n is a real number of from 4 to 30.
(3) A magnetic disk comprising a substrate, at least a recording layer and a protective layer formed on the substrate, and a lubrication layer formed thereon from a compound having a perfluoropolyether structure, the magnetic disk being characterized in that the lubrication layer contains the compound of the formula (I).
(1) Concerning the compound of the invention wherein the end of perfluoropolyether chain is amino group having hydroxyl group
(1)-(1) Synthesis method

Mixed together are perfluoropolyether (wherein the repeated unit is (CF₂CF₂O)m-(CF₂O)n- and both ends are hydroxyl groups), pyridine, dimethylaminopyridine and like basic compounds (the acid component produced as a by-product by the reaction such as CF₃SO₃H being trapped to promote the reaction) and dichloromethane to obtain a solution. Trifluoromethanesulfonic acid anhydride is added dropwise to the solution at room temperature with stirring. The amount of trifluoromethane-sulfonic acid anhydride to be used is at least 2 equivalents of perfluoropolyether. After confirming the terminal point of the reaction by NMR, the contemplated triflate is obtained by extraction from the reaction mixture with perfluorohexane. Then, there are added to the triflate an amine compound having hydroxyl group and, when required, perfluorohexane. The mixture is stirred at a reflux temperature. The amount of the amine compound having hydroxyl group is 2 equivalents or more of the triflate. After confirming by NMR that the triflate used is not left, the reaction is terminated. Thereafter perfluorohexane is added to the reaction mixture. The mixture is washed with dichloromethane and ethanol or water to give the contemplated compound (I). Fomblin Zdol produced by Ausimont S.p.A. can be used as perfluoropolyether having hydroxyl group at each end. The compound has a structure of HO—CH₂CF₂O(CF₂CF₂O)m-(CF₂O)n-CF₂CH₂OH wherein m and n are described above. The compound has a distribution of molecular weight. An average molecular weight is about 1000 to about 7000.

Examples of the amine compound having hydroxyl group are alkanolamine having 1 to 8 carbon atoms, aromatic aminealkanol having 7 to 20 carbon atoms and heteroaminealkanol having 6 to 20 carbon atoms.

Examples of the alkanol amine having 1 to 8 carbon atoms include methanolamine, ethanolamine, propanolamine, butanolamine and hexanolamine. Examples of the aromatic aminealkanol having 7 to 20 carbon atoms include 2-anilinoethanolamine.

Examples of the heteroaminealkanol having 6 to 20 carbon atoms include 1-piperazine ethanol.

(1-2) Amino group having hydroxyl group

The compound of the invention is one wherein at least one end of perfluoropolyether is amino group having hydroxyl group. The amino group having hydroxyl group, namely A, is e.g. diethanolamino, ethylethanolamino, dipropanolamino, 2-anilinoethanol and 1-piperazine ethanol.

(1-3) Amino group having no hydroxyl group or hydroxyl group.

At least one end of perfluoropolyether according to the invention is amino group having hydroxyl group. The other end may be amino group having no hydroxyl group or hydroxyl group. To introduce amino group having no hydroxyl group, an amine compound having no hydroxyl group is combinedly used in an amount of one equivalent or less of the amine compound having hydroxyl group in the synthesis method (1-1).

By the foregoing combined use, a mixture of three components may be provided: the three components being a compound wherein amino group having no hydroxyl group is introduced into both ends of perfluoropolyether, a compound wherein amino group having hydroxyl group is introduced into both ends of perfluoropolyether, and a compound wherein amino group having no hydroxyl group and amino group having hydroxyl group are introduced into each end of perfluoropolyether. In this case, the three-component mixture, as it is, can be used as the lubricant of the present invention. A pure compound containing amino group having no hydroxyl group and amino group having hydroxyl group at each end of perfluoropolyether can be produced by purifying these components by a method such as column chromatography or supercritical carbonic acid extraction.

Examples of the amine compound having no hydroxyl group are aliphatic amine having 1 to 10 carbon atoms, aromatic amine having 6 to 20 carbon atoms, and heteroamine having 4 to 20 carbon atoms.

Examples of the aliphatic amine having 1 to 10 carbon atoms are methylamine, ethylamine, propylamine, butylamine, pentylamine and hexylamine. Examples of the aromatic amine having 6 to 20 carbon atoms are aniline and diphenylamine. Examples of the heteroamine having 4 to 20 carbon atoms include piperazine.

The amino group having no hydroxyl group include, for example, diethylamino, dipropylamino, dibutylamino and diphenylamino.

The symbol m is a real number of 5 to 36, preferably 7 to 25, more preferably 7 to 15. The symbol n is a real number of 4 to 30, preferably 6 to 25, more preferably 6 to 15.

(1-4) Use

The use of the perfluoropolyether compound of the invention includes the application as the lubricant for improving the sliding property of a magnetic disk in a magnetic disk apparatus. This purpose is to reduce the coefficient of the friction between the magnetic disk and the head. Therefore it may be used for other recording devices which involve sliding between the head and the recording medium such as magnetic tapes. The compound of the invention may be used as a lubricant for apparatuses having parts involving sliding as well as for recording devices. The compound of the invention is expected to prevent decomposition of Fomblin-based perfluoropolyether, and may be also used as an additive for a lubricant.

(1-5) Method of use

When a lubrication layer is formed using the perfluoropolyether compound of the invention, it may be applied by a method in which it is applied to the surface as a bulk. However, it may be applied to a thickness more than required. In this case, the lubricant is applied as diluted with a solvent. A fluorine-containing solvent is preferably used in this method in view of compatibility of the solvent with the compound of the invention. Examples of the solvent are HFE-7100 and HFE-7200 (products of 3M), and Vertrel XF (product of DuPont Co. Inc.). When the compound of the invention is used as the lubricant for magnetic disks, magnetic tapes or the like, it is preferably used by a coating method. The compound of the invention can be used also as an additive for a lubricant.

(2) Concerning magnetic disks

(2-1) Structure of magnetic disks

FIG. 1 shows a sectional view schematically showing the magnetic disk of the invention.

The magnetic disk of the invention comprises a substrate 1, at least one recording layer 2 formed on the substrate 1, a protective layer 3 on the recording layer 2 and a lubrication layer 4 formed thereon, as an outermost layer, which contains the compound of the invention.

The substrate 1 is composed of aluminum alloy, glass and like ceramics, polycarbonate or the like.

The recording layer of the magnetic disk, i.e., the magnetic layer is composed of mainly elements capable of forming ferromagnetic bodies, such as iron, cobalt or nickel, alloy or oxide containing chromium, platinum or tantalum in addition to such elements. These materials are applied by, e.g., a plating method or a sputtering method.

The protective layer is formed of carbon, SiC, SiO₂ or the like. The layer is formed mainly by a sputtering method. When the protective layer is formed by a sputtering method, the hardness of the protective layer can be increased by carrying out the method under the atmosphere of Ar/N₂ gas.

By the way, magnetic disks may have projections of about 1 to about 3 nm in height on the surface of the disk or may be flat thereon. It depends on the structure of the magnetic disk apparatus whether the magnetic disk has projections or not. In the case of CSS system, a high adhesion strength is likely to be imposed between the disk and the head on starting the disk so that projections are formed on the disk in this system to reduce the adhesion strength.

On the other hand, in the case of Load/Unload system, adhesion strength is not imposed between the disk and the head on starting the disk so that a flat disk without projections can be used.

(2-2) Method of forming a lubrication layer

Next, the method of forming a lubrication layer will be described. Currently a lubrication layer has a thickness of about 1 to about 2 nm. When perfluoroether is applied as a bulk with an adhesion of about 20 mm²/sec at 40° C., the resulting layer tends to be too thick. In view of this tendency, perfluoroether is used as dissolved in a solvent in application to the underlying layer. Whether the compound of the invention is used as a lubricant, or as an additive for a lubricant, the compound can be applied to the controlled thickness when dissolved in a solvent. The concentration, however, is different depending on the application method, application conditions, component ratio and the like. The application methods to be employed include, for example, a dipping method and a spinning coating.

The solvent to be used is selected from the solvents capable of dissolving perfluoropolyether and the compound of the invention. Specific examples are fluorine-containing solvents such as PF-5060, PF-5080, HFE-7100 and HFE7200 produced by 3M, and Vertrel XF produced by DuPont Co. Inc.

(2-3) Application

The magnetic disk of the invention can be applied to a magnetic disk apparatus which can accommodate the disk and which is provided with a magnetic disk drive including a head for recording, reproducing and erasing information and a motor for rotating the disk; and with a control system for controlling the drive. In such magnetic disk apparatus, the temperature in a hard disk drive is markedly elevated due to heat generated from the motor rotating the magnetic disk. In this state, the head is sliding against the surface of the disk.

The magnetic disk of the invention and the magnetic disk apparatus produced using the magnetic disk thereof can be applied for the following: electronic computers, and outer memories for word processors; and can be also applied in navigation systems, games, cellular phone, PHS (personal handyphone system) and like instruments and machines and inner and outer memories for prevention of crimes in buildings, and for management/control systems of power plants.

<Action>

The perfluoropolyether compound of the invention can reduce the scattering degree of the lubricant and can inhibit decomposition of the lubricant at the same time, whereby the magnetic disk having a lubrication layer containing the lubricant can be provided, and the recording can be reproduced at a high speed by the magnetic disk apparatus fitted with magnetic disks.

The perfluoropolyether compound of the invention has high adsorbability and can reduce the scattering degree of the lubricant presumably for the following reason. Both hydroxyl group and amino group are considered to strongly adsorb to the protective layer on the disk. The decomposition of perfluoropolyether is inhibited presumably because the amino groups existing in the molecules act as a Lewis base and interact with Al₂O₃ ahead of perfluoropolyether.

### BRIEF DESCRIPTION OF THE DRAWING

FIG. 1 is a section view showing the structure of the magnetic disk of the invention. Indicated at 1 is a substrate; at 2, a recording layer; at 3, a protective layer; and at 4, a lubrication layer.

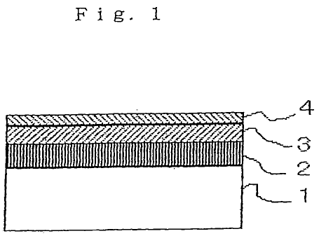

### BEST MODE FOR CARRYING OUT THE INVENITION

The invention will be described in more detail with reference to the following examples to which, however, the invention is not limited.

### EXAMPLE 1

Synthesis of (HOCH₂CH₂)₂N—CH₂CF₂O(CF₂CF₂O)m-(CF₂O)n-CF₂CH₂—N(CH₂CH₂OH)₂ [Compound 1)

Mixed together with stirring were perfluoropolyether having hydroxyl group at an end, i.e. Fomblin Z-DOL (10.0 g) produced by Ausimont S.p.A., pyridine (8.7 g), dimethylaminopyridine (1.8 g) and dichloromethane (50 ml). Trif luoromethanesulf onic acid anhydride (13.9 g) was slowly added. The mixture was continuously stirred at room temperature for 48 hours. After the terminal point of the reaction was confirmed by NMR, the stirring was terminated. Thereafter perf luorohexane (90 ml) was added to the obtained reaction mixture. Then the mixture was washed with a mixed solution of dichloromethane and ethanol. Perfluorohexane was removed by distillation, whereby the contemplated triflate (10.3 g) was produced. The triflate (10.0 g) thus prepared and diethanolamine (8.0 g) were continuously stirred at 105° C. for 48 hours. The terminal point of the reaction was confirmed by NMR and heating and stirring were terminated. Then Vertrel XF (30 ml) was added to the obtained reaction mixture. The mixture was washed with a mixed solution of water and methanol. Vertrel XF was removed by distillation, whereby the contemplated Compound 1 (9.0 g) was produced.

Elementary analysis was not conducted since the Compound 1 did not completely burn. Because the compounds prepared by the following methods (Compounds 2 to 5) failed to completely burn, elementary analysis was not conducted. The reason is presumably that these compounds had too high a content of fluorine.

Therefore only NMR is shown in the analysis results of the compound of the invention.

¹H-NMR (solvent: perfluorohexane, reference material: tetramethylsilane): δ=3.0 ppm [8H, Rf-[CF₂CH₂—N—(CH₂CH₂OH)₂]₂], 3.2 ppm [4H, Rf-[CF₂CH₂—N—(CH₂CH₂OH)₂]₂], 3.6 ppm [8H, Rf-[CF₂CH₂—N—(CH₂CH₂OH)₂]₂], 5.0 ppm [4H, Rf-[CF₂CH₂—N—(CH₂CH₂OH)₂]₂], ¹⁹F-NMR (solvent: none, reference material: OCF₂CF₂CF₂CF₂O in the obtained product being taken as −125.9 ppm): δ=−72.8 ppm, −75.1 ppm (4F, Rf-[CF₂CH₂—N—(CH₂CH₂OH)₂]₂], m=11.1 n=10.0

### EXAMPLE 2

Synthesis of compound having piperazine ethanol at an end [Compound 2]

The triflate (10.0 g) prepared by the method described in Example 1 and 1-piperazine ethanol (6.5 g) were continuously stirred at 105° C. for 48 hours. The terminal point of the reaction was confirmed by NMR, and the heating and stirring were terminated. Then Vertrel XF (30 ml) was added to the obtained reaction mixture. The mixture was washed with a mixed solution of water and methanol. Vertrel XF was removed by distillation, whereby the contemplated Compound 2 (7.2 g) was produced.

The chemical structure of Compound 2 was confirmed by NMR as done in Example 1.

¹H-NMR (solvent: perfluorohexane, reference material: tetramethylsilane): δ=2.4 ppm [8H, Rf-[CF₂CH₂—N═(CH₂CH₂)₂═N—CH₂CH₂OH]₂], 2.4 ppm [4H, Rf-[CF₂CH₂—N═(CH₂CH₂)₂═N—CH₂CH₂OH]₂], 2.8 ppm [8H, Rf-[CF₂CH₂—N═(CH₂CH₂)₂═N—CH₂CH₂OH]₂], 3.6 ppm [4H, Rf-[CF₂CH₂—N═(CH₂CH₂)₂═N—CH₂CH₂OH]₂], 4.0 ppm [4H, Rf-[CF₂CH₂—N═(CH₂CH₂)₂═N—CH₂CH₂OH]₂], ¹⁹F-NMR (solvent: none, reference material: OCF₂CF₂CF₂CF₂O in the obtained product being taken as −125.9 ppm): δ=−73.7 ppm, −75.7 ppm [4F, Rf-[CF₂CH₂—N═(CH₂CH₂)₂═N—CH₂CH₂OH]₂],

In the above, —N═(CH₂CH₂)₂═N— means piperazinyl group. m=9.2 n=11.8

### EXAMPLE 3

Synthesis of (HOCH₂CH₂)₂N—CH₂CF₂O(CF₂CF₂O)m-(CF₂O)n-CF₂CH₂-OH [Compound 3]

The triflate (10.0 g) prepared by the method described in Example 1 and diethanolamine (4.0 g) were continuously stirred at 105° C. for 48 hours. The terminal point of the reaction was confirmed by NMR, and the heating and stirring were terminated. Then Vertrel XF (30 ml) was added to the obtained reaction mixture. The mixture was washed with a mixed solution of water and methanol. After purification by column chromatography, Vertrel XF was removed by distillation, thereby giving the contemplated Compound 3 (2.0 g).

The chemical structure of Compound 3 was confirmed by NMR as done in Example 1.

¹⁹F-NMR (solvent: none, reference material: OCF₂CF₂CF₂CF₂O in the obtained product being taken as −125.9 ppm): δ=−81.3 ppm, −83.3 ppm [2F, Rf-(CF₂CH₂—OH)], δ=−72.8 ppm, −75.1 ppm [2F, Rf-[CF₂CH₂—N—(CH₂CH₂OH)₂]], m=10.0 n=11.0

### EXAMPLE 4

Synthesis of compound having 2-ethylaminoethanol at an end [Compound 4]

The triflate (10.0 g) prepared by the method described in Example 1 and 2-ethylaminoethanol (6.1 g) were continuously stirred at 105° C. for 48 hours. The terminal point of the reaction was confirmed by NMR, and the heating and stirring were terminated. Then Vertrel XF (30 ml) was added to the obtained reaction mixture. The mixture was washed with a mixed solution of water and methanol. Vertrel XF was removed by distillation, whereby the contemplated Compound 4 (6.8 g) was obtained.

The chemical structure of Compound 4 was confirmed by NMR as done in Example 1.

¹H-NMR (solvent: perfluorohexane, reference material: tetramethylsilane): δ=1.2 ppm [6H, Rf-[CF₂CH₂—N(CH₂CH₃)—CH₂CH₂OH]₂], 2.3 ppm [2H, Rf-[CF₂CH₂—N(CH₂CH₃)—CH₂CH₂OH]₂], 2.9 ppm [8H, Rf-[CF₂CH₂—N(CH₂CH₃)—CH₂CH₂OH]₂], 3.2 ppm [4H, Rf-[CF₂CH₂—N(CH₂CH₃)—CH₂CH₂OH]₂], 3.5 ppm [4H, Rf-[CF₂CH₂—N(CH₂CH₃)—CH₂CH₂OH]₂], ¹⁹F-NMR (solvent: none, reference material: OCF₂CF₂CF₂CF₂O in the obtained product being taken as −125.9 ppm): δ=−74.5 ppm, −76.6 ppm [4F, Rf-[CF₂CH₂—N(CH₂CH₃)—CH₂CH₂OH]₂], m=10.9 n=10.5

### COMPARATIVE EXAMPLE 1

(Synthesis of compound described in the specification of U.S. Pat. No.6,083,600) (CH₃CH₂CH₂)₂N—CH₂CF₂O(CF₂CF₂O)m-(CF₂O)n-CF₂CH₂—N(CH₂CH₂CH₃)₂ [Compound 5]

Fomblin Z-DOL produced by Ausimont S.p.A. (10.0 g), namely perfluoropolyether having hydroxyl group at an end, pyridine (8.7 g), dimethylaminopyridine (1.8 g) and dichloromethane (50 ml) were mixed and stirred. To the obtained solution was slowly added trifluoromethanesulfonic acid anhydride (13.9 g). The mixture was continuously stirred at room temperature for 48 hours.

After the terminal point of the reaction was confirmed by NMR, the stirring was terminated. Then after perfluorohexane was added to the obtained reaction mixture, the mixture was washed with a mixed solution of dichloromethane and ethanol. Then perfluorohexane was removed by distillation, whereby the contemplated triflate (10.3 g) was obtained. The obtained triflate (10.0 g) and dipropylamine (7.2 g) were continuously stirred at a reflux temperature for 48 hours. After the terminal point of the reaction was confirmed by NMR, the heating and stirring were terminated. Then after perfluorohexane was added to the obtained reaction mixture, the mixture was washed with a mixed solution of dichloromethane and ethanol. After purification by column chromatography, perfluorohexane was removed by distillation, whereby the contemplated Compound 5 (9.0 g) was produced.

The chemical structure of Compound 5 was confirmed by NMR as done in Example 1.

¹H-NMR (solvent: perfluorohexane, reference material: tetramethylsilane): δ=1.03 ppm [12H, Rf-[CF₂CH₂—N(CH₂CH₂CH₃)₂]₂], 1.58 ppm [8H, Rf-[CF₂CH₂—N(CH₂CH₂CH₃)₂]₂], 2.67 ppm [8H, Rf-[CF₂CH₂—N(CH₂CH₂CH₃)₂]₂], 3.08 ppm [4H, Rf-[CF₂CH₂—N(CH₂CH₂CH₃)₂]₂], ¹⁹F-NMR (solvent: none, reference material: OCF₂CF₂CF₂CF₂O in the obtained product being taken as −125.9 ppm): δ=−74.6 ppm, −76.7 ppm [4F, Rf-[CF₂CH₂—N(CH₂CH₃)₂]₂], m=11.2 n=10.1

### TEST EXAMPLE 1

Measurement of Bond Coefficient

Each of compounds prepared in Examples 1 to 4 and Comparative Example 1 (Compounds 1 to 5) was dissolved in HFE-7100 (product of 3M). The concentration of Compounds 1 to 5 in the solution was 0.1 wt %. Magnetic disks having a diameter of 2.5 inches were immersed in the solution for 1 minute and withdrawn at a rate of 2 mm/s. Thereafter the magnetic disks were placed in a thermostatic oven at 100° C. for 20 minutes and HFE-7100 (solvent) was vaporized. Then an average film thickness of the compound on the disk was measured (film thickness fÅ) by an ellipsometer. Thereafter the disk was immersed in HFE-7100 for 10 minutes and withdrawn at a rate of 4 mm/s. The disk was left to stand at room temperature and the solvent was evaporated. An average film thickness of the compound remaining on the disk was measured (film thickness bÅ) by an ellipsometer. A coefficient of bond generally employed was adopted as an index for indicating the degree of adhesion between the remaining compound and the disk. The coefficient of bond is represented by the following equation:
Coefficient of bond (%)=100 b/f

For comparison, a coefficient of bond was also measured in the same manner as above by using Fomblin Zdol produced by Ausimont S.p.A., i.e., perfluoropolyether having no amino group. Fomblin Zdol (Zdol-4000) is referred to herein as Compound 6.

HOCH₂CF₂O(CF₂CF₂O)m-(CF₂O)n-CF₂CH₂—OH wherein m is 20.0 and n is 19.6. (Compound 6)

The coefficient of bond was measured in respect of these compounds. The results are shown in Table 1. It was confirmed by the results that the perfluoropolyether compound having amino group and hydroxyl group according to the invention exhibited a high adhesion to magnetic disks and were capable of forming a lubrication layer adhered and bonded to the magnetic disk at a high adsorbability.

TABLE 1

| | Bond |
|---|---|
| Specimen | coefficient (%) |
| Compound 1 (compound of Example 1) | 45 |
| Compound 2 (compound of Example 2) | 42 |
| Compound 3 (compound of Example 3) | 45 |
| Compound 4 (compound of Example 4) | 35 |
| Compound 5 (compound of Comparative Example 1) | 21 |
| Compound 6 (Fomblin Zdol-4000) | 17 |

### TEST EXAMPLE 2

Measurement of Resistance to Decomposition by Aluminum Oxide

Specimens were prepared by adding 20 wt % of aluminum oxide (product of Wako Pure Chemical Industries, Ltd., α-alumina, average particle size: 0.5 μm) to each of the compounds produced in Examples 1 to 4 and Comparative Example 1 (Compounds 1 to 5) and Compound 6 and mixing them together by a shaker for 15 minutes or longer.

With use of a thermal analysis apparatus (TG/DTA), comparison was made of the thermal behaviors which differed depending on the presence or absence of aluminum oxide in the specimen. The test was conducted by placing 20 mg of the specimen into a container made of platinum, and heating the specimen at a constant temperature of 250° C. under a nitrogen atmosphere. The change in the reduction of weight of the compound with time was checked.

The results are shown in Table 2. It was confirmed as apparent from Table 2 that the perfluoropolyether compound of the invention was excellent in resistance to decomposition by aluminum oxide.

TABLE 2

| | Ratio of decrease in weight (%) | | | |
|---|---|---|---|---|
| | Absence of alumina | | Presence of alumina | |
| | After | After | After | After |
| Specimen | 100 min | 200 min | 100 min | 200 min |
| Compound 1 | 5 | 8 | 6 | 11 |
| (compound of Example 1) | | | | |
| Compound 2 | 5 | 9 | 7 | 11 |
| (compound of Example 2) | | | | |
| Compound 3 | 6 | 9 | 7 | 12 |
| (compound of Example 3) | | | | |
| Compound 4 | 4 | 10 | 6 | 8 |
| (compound of Example 4) | | | | |
| Compound 5 | 10 | 20 | 11 | 23 |
| (compound of | | | | |
| Comparative Example 1) | 15 | 28 | 33 | 100 |
| Compound 6 (Fomblin | | | | |
| Zdol - 4000) | | | | |

### TEST EXAMPLE 3

A solution of each of Compounds 1 to 4 in HFE-7100 at a concentration of 0.1 wt % was prepared in the same manner as in Test Example 1. A carbon layer (film thickness about 15 nm) was formed by a sputtering deposition method on the surface of a hard disk having a magnetic layer on the surface of a disk of Al alloy of 3 in. in diameter. The carbon layer is a DLC protective layer. The disk was immersed in the HFE solution for 1 minute and withdrawn at a rate of 2 mm/s. A magnetic disk was produced by forming a lubrication layer of each of Compounds 1 to 3 on the surface by being left in a thermostatic oven at 100° C. for 20 minutes. The lubrication layer had an average film thickness of 20 to 25 Å. For comparison, a magnetic disk having a lubrication layer of Compound 6 on the surface was produced. The lubrication layer was formed in the same manner as in the formation of the lubrication layers of Compounds 1 to 4 except that only the concentration in the solution was different (0.2 wt %) . The lubrication layer thus formed had an average film thickness of 22Å.

The magnetic disk thus made was fixed to a CSS tester fitted with lamp load means. The head was made ready outside the disk while the disk was continuously rotated for 1000 hours at 12,000 rpm. Then the head was moved onto the surface of the disk and was made to slide against the disk at a head load of 2 g and at a rotational frequency of 12,000 rpm. The friction force during rotation was measured in this way.

The magnetic disks coated with each of the Compounds 1 to 4 showed a friction force of 2 g or less, whereas the magnetic disk coated with the Compound 6 showed a friction force of 5 g or more.

The surface of these magnetic disks was observed under a microscope. The disk coated with the Compound 6 indicated a trace of sliding at a contact portion between the disk and the head. This means that the DLC protective layer was removed. No tracing of sliding was observed on the surface of the disk coated with each of the Compounds 1 to 4.

Investigation was carried out in respect of the thickness of the lubrication layer after rotation of the disks for 1000 hours. The lubrication layers of Compounds 1 to 4 retained 80% or higher (18 Å or higher) of the initial film thickness after rotation for 1000 hours. However, the lubrication layer of Compound 6 had a thickness reduced to 30% or lower (6 Å or lower) of the initial film thickness after rotation for 1000 hours. The reduction in the thickness of the lubrication layer is attributed to scattering of the compound constituting the lubrication layer due to rotation of the magnetic disk. When an average film thickness is decreased to about 6 Å, there is formed part of the surface of the lubrication layer where the compound does not exist at all. Namely the lubrication layer becomes partly defective and becomes markedly impaired in lubricity, presumably resulting in increased friction force.

Elementary analysis was performed by XPS (X-ray Photoelectron Spectroscopy) as to the sliding surface of the head after 1000-hour rotation. A signal derived from bonding of Al-F was strongly seen as to the head having slidden against the disk having the lubrication layer of Compound 5. It was suggested that perfluoroether was reacted with Al on the surface of the head and was decomposed. On the other hand, on the head having slidden against the disk having a lubrication layer of each of Compounds 1 to 4, a signal derived from bonding of Al-F was not detected. Presumably unlike the Compound 6, Compounds 1 to 4 are such that perfluoropolyether chain in the molecule is unlikely to decompose due to Al compound in the head material.

The above results show that the perfluoropolyether compound having amino group and hydroxyl group according to the invention can effectively act as a low scattering-degree lubricant for a magnetic disk.

### Industrial Applicability

The compound of the invention which has amino group having hydroxyl group at an end of perfluoropolyether chain can provide a lubricant which can achieve two substantially non-achievable objects of lowering the scattering degree of the lubricant and inhibiting the decomposition of the lubricant at the same time. The magnetic disk containing the compound of the invention as the lubricant can resist continuous rotation of the magnetic disk at a high rotational speed.

1. A compound of the formula (I)
A—CH₂CF₂O(CF₂CF₂O)m-(CF₂O)n-CF₂CH₂—B (I)
wherein A is amino group having hydroxyl group, B is hydroxyl group, or amino group having or not having hydroxyl group, wherein m is a real number of 5 to 36, and n is a real number of from 4 to 30.

2. A compound as defined in claim 1 wherein the amino group having. hydroxyl group is one in which hydrogen atom bonded to nitrogen atom is removed from alkanolamine having 1 to 8 carbon atoms, aromatic aminealkanol having 7 to 20 carbon atoms or heteroaminealkanol having 6 to 20 carbon atoms.

3. A compound as defined in claim 2 wherein the amino group having hydroxyl group is diethanolamino, ethylethanolamino, dipropanolamino, 2-anilinoethanol or 1-piperazine ethanol.

4. A compound as defined in claim 1 wherein the amino group having no hydroxyl group is one in which hydrogen atom bonded to nitrogen atom is removed from aliphatic amine having 1 to 10 carbon atoms, aromatic amine having 6 to 20 carbon atoms, or heteroamine having 4 to 20 carbon atoms.

5. A compound as defined in claim 4 wherein the amino group having no hydroxyl group is diethylamino, dipropylamino, dibutylamino or diphenylamino.

6. A lubricant containing the compound of the formula (I)
A—CH₂CF₂O(CF₂CF₂O)m-(CF₂O)n-CF₂CH₂—B (I)
wherein A is amino group having hydroxyl group, B is hydroxyl group, or amino group having or not having hydroxyl group, wherein m is a real number of 5 to 36, and n is a real number of from 4 to 30.

7. A lubricant as defined in claim 6 wherein the amino group having hydroxyl group is diethanolamino, ethylethanolamino, dipropanolamino, 2-anilinoethanol or 1-piperazine ethanol.

8. A lubricant as defined in claim 6 wherein the amino group having no hydroxyl group is dipropylamino, dibutylamino, diethylamino, dibenzoamino or diphenylamino.

9. A magnetic disk comprising a substrate, at least a recording layer and a protective layer formed on the substrate, and a lubrication layer formed thereon from a compound having a perfluoropolyether structure, the magnetic disk being characterized in that the lubrication layer contains the compound of the formula (I)
A—CH₂CF₂O(CF₂CF₂O)m-(CF₂O)n-CF₂CH₂—B (I)
wherein A is amino group having hydroxyl group, B is hydroxyl group, or amino group having or not having hydroxyl group, wherein m is a real number of 5 to 36, and n is a real number of from 4 to 30.
